(19) United States
(12) Patent Application Publication
(10) Pub. No.: US 2026/0120823 A1
(43) Pub. Date: Apr. 30, 2026

(54) GENERATIVE-DISCRIMINATIVE ARTIFICAL-INTELLIGENCE FRAMEWORK FOR DIGITAL-TWIN DISCOVERY OF THIN-FILM MATERIALS

(71) Applicant: DEEP FOREST SCIENCES, INC., Palo Alto, CA (US)

(72) Inventors: BHARATH RAMSUNDAR, Palo Alto, CA (US); KARAN BANIA, Jamnagar (IN); ARYAN AMIT BARSAINYAN, Bellary (IN); SHREYAS VINAYA SATHYANARAYANA, Bangalore (IN)

(73) Assignee: DEEP FOREST SCIENCES, INC., Palo Alto, CA (US)

(21) Appl. No.: 19/368,900

(22) Filed: Oct. 24, 2025

Related U.S. Application Data
(60) Provisional application No. 63/711,488, filed on Oct. 24, 2024.

(51) Int. Cl. G16C 60/00; G16C 20/30; G16C 20/70

(52) U.S. Cl. CPC G16C 60/00; G16C 20/30; G16C 20/70

(57) ABSTRACT

Various aspects of the present disclosure relate to techniques for generative-discriminative artificial-intelligence framework for digital-twin discovery of thin-film materials. An apparatus is configured to generate, using a generative artificial intelligence model, one or more precursor molecular structures for forming a thin film; simulate, using a digital-twin model of a thin-film deposition process, formation of the thin film from the one or more precursor molecular structures; determine, from results of the simulation, one or more physical properties of the thin film; train a discriminative model using the one or more physical properties of the thin film, wherein the discriminative model learns to predict thin-film properties without requiring full simulation; and update the generative artificial intelligence model based on the one or more physical properties of the thin film or predictions generated by the discriminative model to iteratively improve discovery of materials having target dielectric characteristics.

### CROSS REFERENCE TO RELATED APPLICATIONS

This application claims the benefit of U.S. Provisional Patent Application No. 63/711,488 entitled “TECHNIQUES FOR GENERATIVE DISCRIMINATIVE PIPELINE FOR LOW-K DIELECTRICS” and filed on Oct. 24, 2024, for Bharath Ramsundar, et al., which is incorporated herein by reference

### FIELD

The subject matter herein relates generally to materials science and computational modeling, and more particularly to systems and methods that employ artificial intelligence and digital-twin simulation for autonomous discovery, design, and optimization of thin-film materials.

### BACKGROUND

Discovery and optimization of new materials are fundamental to advancements in electronics, energy storage, coatings, and manufacturing. Traditional experimental approaches to materials design rely on slow and costly trial-and-error processes, while purely computational methods such as density-functional theory (DFT) are limited by high computational cost and scale poorly for complex, disordered systems. Recent developments in artificial intelligence and machine learning have enabled data-driven exploration of vast chemical and structural spaces, yet most existing approaches operate as static predictors rather than adaptive design systems.

### SUMMARY

In one embodiment, an apparatus is configured to generate, using a generative artificial intelligence model, one or more precursor molecular structures for forming a thin film; simulate, using a digital-twin model of a thin-film deposition process, formation of the thin film from the one or more precursor molecular structures; determine, from results of the simulation, one or more physical properties of the thin film; train a discriminative model using the one or more physical properties of the thin film, wherein the discriminative model learns to predict thin-film properties without requiring full simulation; and update the generative artificial intelligence model based on the one or more physical properties of the thin film or predictions generated by the discriminative model to iteratively improve discovery of materials having target dielectric characteristics.

In one embodiment, a method is configured for generating, using a generative artificial intelligence model, one or more precursor molecular structures for forming a thin film; simulating, using a digital-twin model of a thin-film deposition process, formation of the thin film from the one or more precursor molecular structures; determining, from results of the simulation, one or more physical properties of the thin film; training a discriminative model using the one or more physical properties of the thin film, wherein the discriminative model learns to predict thin-film properties without requiring full simulation; and updating the generative artificial intelligence model based on the one or more physical properties of the thin film or predictions generated by the discriminative model to iteratively improve discovery of materials having target dielectric characteristics.

In one embodiment, a computer program product is embodied on a non-transitory computer-readable medium comprising instructions that, when executed by a processor, cause the processor to perform operations comprising generating, using a generative artificial intelligence model, one or more precursor molecular structures for forming a thin film; simulating, using a digital-twin model of a thin-film deposition process, formation of the thin film from the one or more precursor molecular structures; determining, from results of the simulation, one or more physical properties of the thin film; training a discriminative model using the one or more physical properties of the thin film, wherein the discriminative model learns to predict thin-film properties without requiring full simulation; and updating the generative artificial intelligence model based on the one or more physical properties of the thin film or predictions generated by the discriminative model to iteratively improve discovery of materials having target dielectric characteristics.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a schematic block diagram illustrating one embodiment of a system in accordance with the subject matter disclosed herein;

FIG. 2 illustrates one example of an apparatus in accordance with the subject matter disclosed herein;

FIG. 3 illustrates a generative discriminative pipeline in accordance with the subject matter disclosed herein;

FIG. 4 illustrates an example embodiment showing correspondence between an experimental thin-film deposition technique and its digital-twin simulation accordance with the subject matter disclosed herein;

FIG. 5 illustrates an example implementation of motif-clustering and dielectric-estimation stage in accordance with the subject matter disclosed herein;

FIG. 6 illustrates a flowchart showing one example of a method in accordance with the subject matter disclosed herein; and

FIG. 7 illustrates a flowchart showing one example of a method in accordance with the subject matter disclosed herein.

### DETAILED DESCRIPTION

The embodiments described herein provide a unified framework for the discovery and optimization of thin-film materials through the combined use of generative artificial intelligence and physics-based simulation. The claimed solution addresses a long-standing challenge in materials science: predicting and designing complex, non-homogeneous thin films such as low-k dielectrics, which are critical for semiconductor interconnects and other high-performance electronic components.

In many embodiments, the present framework is applied to the discovery and optimization of thin-film materials used in semiconductor, optical, and energy devices. A thin film, as used herein, refers to a layer of material having a thickness ranging from a few nanometers to several micrometers deposited on a substrate to modify its electrical, mechanical, optical, or chemical properties. Thin films are fundamental to integrated-circuit fabrication, interconnect insulation, barrier formation, and surface passivation.

Thin-film behavior differs markedly from bulk materials. Atomic-scale interfaces, porosity, and compositional gradients strongly influence properties such as dielectric constant, leakage current, adhesion strength, and mechanical hardness. Because these films are often amorphous or nanocrystalline, their physical properties cannot be derived from ideal crystal models alone. Conventional ab-initio simulations that assume homogeneous periodic structures fail to capture local disorder and film-growth kinetics, while empirical experimentation remains slow and expensive.

In one example application, the disclosed system is used for low-k dielectric thin films. The “k” value, or relative permittivity, measures a film's ability to store electrical charge. Reducing the k-value in inter-metal dielectrics lowers parasitic capacitance and signal delay in microelectronic interconnects. However, achieving low k-values often compromises mechanical integrity, as highly porous films tend to be fragile. The design challenge thus involves balancing electrical and mechanical performance while ensuring process compatibility with standard deposition tools.

In traditional plasma-enhanced chemical-vapor-deposition (PECVD) processes, a molecular precursor gas is introduced into a reaction chamber where it dissociates under plasma excitation, forming reactive species that adsorb and polymerize on a heated substrate. The resulting film's density, porosity, and stoichiometry depend on numerous parameters, including precursor composition, plasma power, pressure, and substrate temperature. Small changes in any of these variables can dramatically alter the resulting dielectric constant and hardness.

The digital-twin model described herein captures these complex dependencies by representing both the gas-phase precursor reactions and surface-growth dynamics in silico. The model computes reaction energetics, diffusion rates, and local bonding configurations, yielding an atomistic reconstruction of the growing film. The motif-based voxelization approach further divides the simulated film into discrete atomic neighborhoods so that spatial variations in density or composition can be analyzed. This enables the system to mimic non-uniformities such as gradient porosity or local cross-linking observed in experimental PECVD films.

Although low-k dielectrics represent one exemplary target, the same generative-discriminative framework applies to other thin-film categories, including:
High-k gate oxides (e.g., hafnium oxide, zirconium oxide) for transistor gate stacks;
Conductive or barrier layers (e.g., ruthenium, tantalum nitride) used in interconnects;
Optical coatings for reflectivity or antireflection control; and
Protective and catalytic films for corrosion resistance or energy applications.

For each of these, the system's generative module can propose novel precursor molecules or alloy compositions, while the digital-twin simulator models nucleation, grain formation, and defect incorporation relevant to the chosen deposition technique. The discriminative and feedback modules then optimize toward target physical properties, such as refractive index, bandgap, resistivity, or mechanical modulus, depending on the application.

Accordingly, the term “thin film” as used throughout this disclosure should be understood to include dielectric, conductive, semiconductive, or composite films formed by vapor-phase or solution-based deposition methods. The disclosed solution is capable of simulating and optimizing any such film by coupling artificial-intelligence-driven design with physics-based process modeling.

In conventional design workflows, researchers rely on empirical screening or ab-initio computational techniques such as density functional theory (DFT) to estimate material properties. However, these approaches scale poorly for thin films, which may contain tens of thousands of atoms and exhibit structural variation across multiple length scales. Simulating such systems directly requires supercomputing resources and cannot feasibly be coupled to iterative design loops. Furthermore, purely discriminative machine learning models can predict dielectric constants or related properties only for known compositions; they cannot propose new precursor chemistries or simulate the deposition process that forms the film itself.

The present framework overcomes these limitations by coupling a generative model, a digital-twin physical simulator, and a discriminative evaluation model within a closed-loop feedback system. The generative model—implemented using a conditional variational autoencoder (CDVAE) or other deep generative network—produces candidate precursor molecular structures that could yield desirable dielectric properties. These candidates are provided to a digital-twin simulator that approximates the physical process of thin-film formation, such as PECVD. The simulator computes film-level physical properties, including dielectric constant, hardness, and density, based on molecular inputs and process parameters.

To accelerate these computations, the system performs motif-based voxelization of the simulated film structure. The atomic model is divided into three-dimensional voxel elements, and representative motifs are identified using clustering algorithms such as smooth overlap of atomic positions (SOAP) descriptors. Physical simulations are then performed on the representative motifs rather than the entire film, producing a rapid and accurate proxy for large-scale film behavior. This motif-based digital twin captures both the chemical and morphological heterogeneity inherent in real deposited materials.

The resulting simulated properties are evaluated by a discriminative model—such as a random forest regressor or a graph neural network (e.g., SchNet)—that predicts dielectric performance and other key metrics. Deviations between the simulated results and target properties are used as feedback to retrain the generative model, thereby biasing future generations toward improved candidates. In certain embodiments, the framework further incorporates vibrational effects through density functional perturbation theory (DFPT) and may integrate domain knowledge from large language models (LLMs) trained on patent and scientific literature describing dielectric materials.

Through iterative operation of these interconnected components, the system progressively converges on precursor and process combinations predicted to yield thin films with target dielectric properties. This integration of generative AI, digital-twin physics modeling, and discriminative evaluation establishes a new computational paradigm for autonomous materials discovery and optimization.

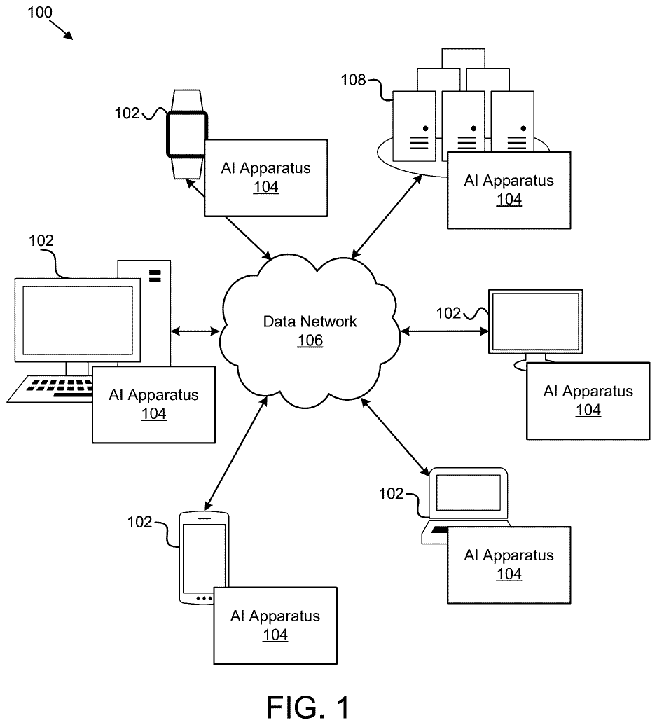

FIG. 1 is a schematic block diagram illustrating one embodiment of a system 100 for techniques for generative discriminative pipeline for low-k dielectrics. In one embodiment, the system 100 includes one or more information handling devices 102, one or more AI apparatuses 104, one or more data networks 106, and one or more servers 108. In certain embodiments, even though a specific number of information handling devices 102, AI apparatuses 104, data networks 106, and servers 108 are depicted in FIG. 1, one of skill in the art will recognize, in light of this disclosure, that any number of information handling devices 102, AI apparatuses 104, data networks 106, and servers 108 may be included in the system 100.

In one embodiment, the system 100 includes one or more information handling devices 102. The information handling devices 102 may be embodied as one or more of a desktop computer, a laptop computer, a tablet computer, a smart phone, a smart speaker (e.g., Amazon Echo®, Google Home®, Apple HomePod®), an Internet of Things device, a security system, a set-top box, a gaming console, a smart TV, a smart watch, a fitness band or other wearable activity tracking device, an optical head-mounted display (e.g., a virtual reality headset, smart glasses, head phones, or the like), a High-Definition Multimedia Interface (“HDMI”) or other electronic display dongle, a personal digital assistant, a digital camera, a video camera, or another computing device comprising a processor (e.g., a central processing unit (“CPU”), a processor core, a field programmable gate array (“FPGA”) or other programmable logic, an application specific integrated circuit (“ASIC”), a controller, a microcontroller, and/or another semiconductor integrated circuit device), a volatile memory, and/or a non-volatile storage medium, a display, a connection to a display, and/or the like.

The thin-film design and optimization workflow described herein may be implemented by a computer-based architecture that integrates data-driven and physics-based models within a unified artificial-intelligence framework. In one embodiment, this architecture is realized as an AI apparatus 104 configured to perform the generative, simulative, and evaluative operations discussed herein. The AI apparatus 104 executes a series of cooperative modules that correspond to the functional stages of the framework, including the generation of precursor structures, simulation of thin-film formation, evaluation of physical properties, and iterative retraining based on feedback. Each module described below may be implemented as hardware, software, firmware, or a combination thereof, and collectively they enable automated discovery and optimization of thin-film materials for electronic, optical, and energy applications.

In certain embodiments, the AI apparatus 104 may include a hardware device such as a secure hardware dongle or other hardware appliance device (e.g., a set-top box, a network appliance, or the like) that attaches to a device such as a head mounted display, a laptop computer, a server 108, a tablet computer, a smart phone, a security system, a network router or switch, or the like, either by a wired connection (e.g., a universal serial bus (“USB”) connection) or a wireless connection (e.g., Bluetooth®, Wi-Fi, near-field communication (“NFC”), or the like); that attaches to an electronic display device (e.g., a television or monitor using an HDMI port, a DisplayPort port, a Mini DisplayPort port, VGA port, DVI port, or the like); and/or the like. A hardware appliance of the AI apparatus 104 may include a power interface, a wired and/or wireless network interface, a graphical interface that attaches to a display, and/or a semiconductor integrated circuit device as described below, configured to perform the functions described herein with regard to the AI apparatus 104.

The AI apparatus 104, in such an embodiment, may include a semiconductor integrated circuit device (e.g., one or more chips, die, or other discrete logic hardware), or the like, such as a field-programmable gate array (“FPGA”) or other programmable logic, firmware for an FPGA or other programmable logic, microcode for execution on a microcontroller, an application-specific integrated circuit (“ASIC”), a processor, a processor core, or the like. In one embodiment, the AI apparatus 104 may be mounted on a printed circuit board with one or more electrical lines or connections (e.g., to volatile memory, a non-volatile storage medium, a network interface, a peripheral device, a graphical/display interface, or the like). The hardware appliance may include one or more pins, pads, or other electrical connections configured to send and receive data (e.g., in communication with one or more electrical lines of a printed circuit board or the like), and one or more hardware circuits and/or other electrical circuits configured to perform various functions of the AI apparatus 104.

The semiconductor integrated circuit device or other hardware appliance of the AI apparatus 104, in certain embodiments, includes and/or is communicatively coupled to one or more volatile memory media, which may include but is not limited to random access memory (“RAM”), dynamic RAM (“DRAM”), cache, or the like. In one embodiment, the semiconductor integrated circuit device or other hardware appliance of the AI apparatus 104 includes and/or is communicatively coupled to one or more non-volatile memory media, which may include but is not limited to: NAND flash memory, NOR flash memory, nano random access memory (nano RAM or “NRAM”), nanocrystal wire-based memory, silicon-oxide based sub-10 nanometer process memory, graphene memory, Silicon-Oxide-Nitride-Oxide-Silicon (“SONOS”), resistive RAM (“RRAM”), programmable metallization cell (“PMC”), conductive-bridging RAM (“CBRAM”), magneto-resistive RAM (“MRAM”), dynamic RAM (“DRAM”), phase change RAM (“PRAM” or “PCM”), magnetic storage media (e.g., hard disk, tape), optical storage media, or the like.

The data network 106, in one embodiment, includes a digital communication network that transmits digital communications. The data network 106 may include a wireless network, such as a wireless cellular network, a local wireless network, such as a Wi-Fi network, a Bluetooth® network, a near-field communication (“NFC”) network, an ad hoc network, and/or the like. The data network 106 may include a wide area network (“WAN”), a storage area network (“SAN”), a local area network (“LAN”) (e.g., a home network), an optical fiber network, the internet, or other digital communication network. The data network 106 may include two or more networks. The data network 106 may include one or more servers, routers, switches, and/or other networking equipment. The data network 106 may also include one or more computer readable storage media, such as a hard disk drive, an optical drive, non-volatile memory, RAM, or the like.

The wireless connection may be a mobile telephone network. The wireless connection may also employ a Wi-Fi network based on any one of the Institute of Electrical and Electronics Engineers (“IEEE”) 802.11 standards. Alternatively, the wireless connection may be a Bluetooth® connection. In addition, the wireless connection may employ a Radio Frequency Identification (“RFID”) communication including RFID standards established by the International Organization for Standardization (“ISO”), the International Electrotechnical Commission (“IEC”), the American Society for Testing and Materials® (ASTM®), the DASH7™ Alliance, and EPCGlobal™.

Alternatively, the wireless connection may employ a ZigBee® connection based on the IEEE 802 standard. In one embodiment, the wireless connection employs a Z-Wave® connection as designed by Sigma Designs®. Alternatively, the wireless connection may employ an ANT® and/or ANT+® connection as defined by Dynastream® Innovations Inc. of Cochrane, Canada.

The wireless connection may be an infrared connection including connections conforming at least to the Infrared Physical Layer Specification (“IrPHY”) as defined by the Infrared Data Association® (“IrDA”®). Alternatively, the wireless connection may be a cellular telephone network communication. All standards and/or connection types include the latest version and revision of the standard and/or connection type as of the filing date of this application.

The one or more servers 108, in one embodiment, may be embodied as blade servers, mainframe servers, tower servers, rack servers, and/or the like. The one or more servers 108 may be configured as mail servers, web servers, application servers, FTP servers, media servers, data servers, web servers, file servers, virtual servers, and/or the like. The one or more servers 108 may be communicatively coupled (e.g., networked) over a data network 106 to one or more information handling devices 102 and may be configured to execute or run machine learning algorithms, programs, applications, processes, and/or the like; communicate with a thermal imaging device; store thermal imaging data in a database, blockchain, or other secure data structure; and/or the like.

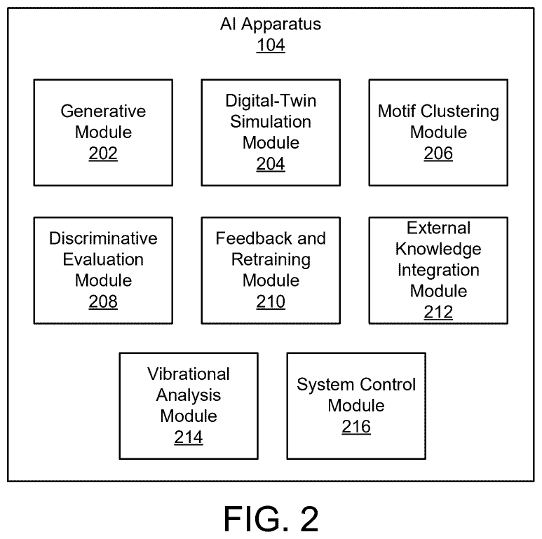

FIG. 2 depicts one embodiment of an apparatus for generative-discriminative artificial-intelligence framework for digital-twin discovery of thin-film materials. In one embodiment, the apparatus includes an instance of an AI apparatus 104. The AI apparatus 104, in one embodiment, includes one or more of a generative module 202, a digital-twin simulation module 204, a motif clustering module 206, a discriminative evaluation module 208, a feedback and retraining module 210, an external knowledge integration module 212, a vibrational analysis module 214, and a system control module 216. Each module may be implemented as software instructions executed by one or more processors, hardware logic, firmware, or any combination thereof.

In one embodiment, the generative module 202 is configured to generate one or more precursor molecular structures for potential thin-film materials. In one embodiment, the generative module 202 employs a conditional variational autoencoder (CDVAE) trained to generate candidate molecules conditioned on target dielectric properties. The generative module 202 may alternatively employ other deep generative models such as diffusion models, graph-based molecular generators, or LLMs fine-tuned for chemical synthesis. In one embodiment, the generative module 202 updates the prompt and embedding context of the generative large-language model using structure-property correlations returned from prior discriminative evaluations.

In one embodiment, the generative module 202 outputs molecular representations including atomic coordinates, bonding information, and relevant process parameters such as precursor gas composition or deposition temperature. These representations are transmitted to the digital-twin simulation module 204 for physical modeling.

In some embodiments, the generative module 202 incorporates transfer learning, allowing the generator to adapt to new chemical domains as additional data is obtained from simulated or experimental results. The generator may also interface with external knowledge integration module 212 to ingest domain-specific literature or prior patent data relevant to dielectric materials.

In one embodiment, the digital-twin simulation module 204 models physical formation of a thin film from precursors generated by module 202. The digital-twin reproduces, in silico, the plasma-enhanced chemical-vapor-deposition (PECVD) process or other deposition techniques such as atomic-layer deposition (ALD).

The simulation may include parameters such as gas composition, plasma energy, substrate temperature, deposition rate, and mean-free-path of reactive species. These parameters govern precursor decomposition, surface binding, and film densification. Empirical calibration constants, derived from measured deposition data, can be incorporated to align simulated film densities with experimental results.

The digital-twin simulation module 204, in one embodiment, calculates film-level properties including density, hardness, dielectric constant, and porosity by combining classical molecular-dynamics with quantum-mechanical solvers such as DFT. Because full DFT simulation of heterogeneous films is computationally prohibitive, the digital-twin simulation module 204 cooperates with the motif-clustering module 206 to implement a motif-based approximation, preserving accuracy while reducing compute cost. In one embodiment, the digital-twin simulation module 204 may cache intermediate atomic configurations to accelerate retraining and reuse previously equilibrated structures.

In one embodiment, the motif-clustering module 206 partitions the simulated film structure into three-dimensional volumetric elements or voxels. Each voxel is characterized by a SOAP descriptor or equivalent invariant representation encoding local atomic environments.

The motif-clustering module 206 computes embedding vectors for each voxel and applies similarity metrics such as cosine distance or SOAP-kernel distance to group similar voxels into motifs representing unique atomic configurations. Each cluster centroid becomes a representative motif on which high-fidelity physics simulations are performed.

After motif-level simulations, the motif-clustering module 206 performs weighted reassembly or spatial averaging to reconstruct global film properties, producing accurate approximations at a fraction of the computational cost of full-film DFT. This motif-based approach captures structural heterogeneity and enables efficient coupling to the feedback loop.

In one embodiment, the discriminative evaluation module 208 receives simulation outputs and predicts material properties using one or more discriminative models. In one embodiment, evaluation is hierarchical—a Random-Forest regressor acts as a rapid pre-filter to discard low-quality candidates, and a graph-neural-network model (such as SchNet) refines predictions of dielectric constant, hardness, and mechanical density.

The discriminative evaluation module 208, in one embodiment, produces uncertainty estimates or confidence intervals that guide active learning and prioritization of candidates. Interpretability tools such as SHapley Additive explanations (SHAP) values or gradient-based saliency maps reveal which atomic or process features most influence dielectric performance. Feature-importance rankings are stored for human review and for retraining of the generative model. Once trained, the discriminative model can predict thin-film dielectric and mechanical properties directly from motif-level or process descriptors without executing a full digital-twin simulation.

In one embodiment, the feedback and retraining module 210 forms the closed-loop control mechanism of the system. The feedback and retraining module 210 computes a multi-objective reward function incorporating deviations between simulated dielectric constants and target values, mechanical-property penalties, and computational efficiency metrics.

In one embodiment, the feedback and retraining module 210 updates weights of the generative model 202 using gradient-descent or reinforcement-learning techniques to bias future generations toward improved candidates. Convergence is monitored by tracking moving averages of reward values, and adaptive sampling adjusts perturbation magnitude or training frequency when improvement thresholds plateau.

Through repeated execution, the feedback and retraining module 210 enables self-optimizing exploration of the chemical design space without human intervention.

In one embodiment, the external-knowledge module 212 augments the framework with scientific and patent information. The external-knowledge module 212 maintains a vector database of documents encoded as semantic embeddings generated by an LLM trained on materials-science corpora. When a new precursor is proposed, the external-knowledge module 212 queries the database using its embedding to retrieve related compositions, deposition conditions, and empirical dielectric data.

Extracted insights—such as known synthesis constraints or reported property ranges—are converted into conditioning vectors or penalty terms for the generative module 202. This ensures that generated candidates remain chemically feasible and guided by collective prior knowledge.

In one embodiment, the vibrational-analysis module 214 refines property predictions by incorporating vibrational and phonon effects often neglected in static DFT simulations. Using DFPT or equivalent perturbative methods, the vibrational-analysis module 214 computes phonon dispersion relations and electron-phonon coupling constants.

The resulting vibrational-correction term Ak_vib is added to the electronic dielectric constant predicted by the discriminative model 208 to produce a corrected total dielectric constant. This DFPT branch operates in parallel with the digital-twin simulator and provides fine-grained corrections especially relevant for amorphous or semi-crystalline films.

In one embodiment, the system-control module 216 orchestrates workflow execution and manages all generated data. It stores precursor identifiers, simulation parameters, motif embeddings, and computed properties in a relational or graph-based database. Version control ensures full traceability of each design iteration.

The system-control module 216 implements an active-learning scheduler that prioritizes candidates with high predictive uncertainty to maximize information gain. Results are continuously aggregated into training datasets for the generative module 202 and the discriminative evaluation module 208. Visualization and reporting interfaces allow researchers to review simulation outcomes or export candidates for experimental validation.

An example application of the disclosed system is the design of low-k dielectric thin films for semiconductor interconnect structures. The term “low-k dielectric” refers to a material having a relative permittivity (k value) less than that of silicon dioxide (k≈4.0). Reducing the dielectric constant of inter-metal dielectrics decreases parasitic capacitance, cross-talk, and signal delay between adjacent interconnect lines, thereby improving circuit speed and lowering power consumption in integrated circuits.

Modern low-k films typically employ organosilicate or carbon-doped oxide chemistries formed by PECVD or spin-on processes. Their microstructure often contains nanoscale pores or organic bridges that disrupt the continuous polar Si—O—Si network, thereby reducing polarizability. However, these same modifications can reduce mechanical strength and lead to plasma or moisture damage during subsequent processing steps. Balancing dielectric constant, density, hardness, and chemical stability remains a core materials-science challenge.

The solutions described herein directly address this challenge by coupling generative design and physics-based simulation in a single automated framework. The generative module 202 explores chemical design space to propose novel precursors capable of forming films with intrinsically low polarizability, controlled porosity, and stable cross-linking networks. Candidate structures may include modified silsesquioxanes, organosilicon cages, or hybrid inorganic-organic precursors that have not been previously synthesized.

The digital-twin simulation module 204 then models the PECVD process that transforms each proposed precursor into a solid film. Plasma power, gas flow rate, and substrate temperature are varied to predict how precursor fragmentation and recombination influence film density and composition. The resulting atomistic film structures are partitioned by the motif-clustering module 206 to capture nanoscale heterogeneity such as pores or carbon-rich domains that dominate the effective dielectric constant.

The discriminative evaluation module 208 computes or predicts key film properties—dielectric constant (k), hardness, density, and elastic modulus—and identifies optimal trade-offs among these parameters. Through the feedback and retraining module 210, the generative model learns which precursor motifs and process conditions most effectively achieve target k values (e.g., below 3.0 or even 2.5) without sacrificing mechanical robustness.

In certain embodiments, the framework also incorporates DFPT calculations via vibrational-analysis module 214 to capture phonon-driven polarization effects that influence the measured dielectric constant of amorphous films. Additionally, external-knowledge module 212 enables integration of empirical data from scientific publications and patents describing known low-k materials, providing chemical priors that accelerate convergence of the generative model.

Collectively, these modules create a closed-loop digital-twin environment that can autonomously propose, simulate, and refine new low-k dielectric materials before laboratory synthesis. This approach significantly reduces experimental trial-and-error and opens pathways to ultralow-k films that maintain high mechanical strength and process compatibility for advanced semiconductor nodes.

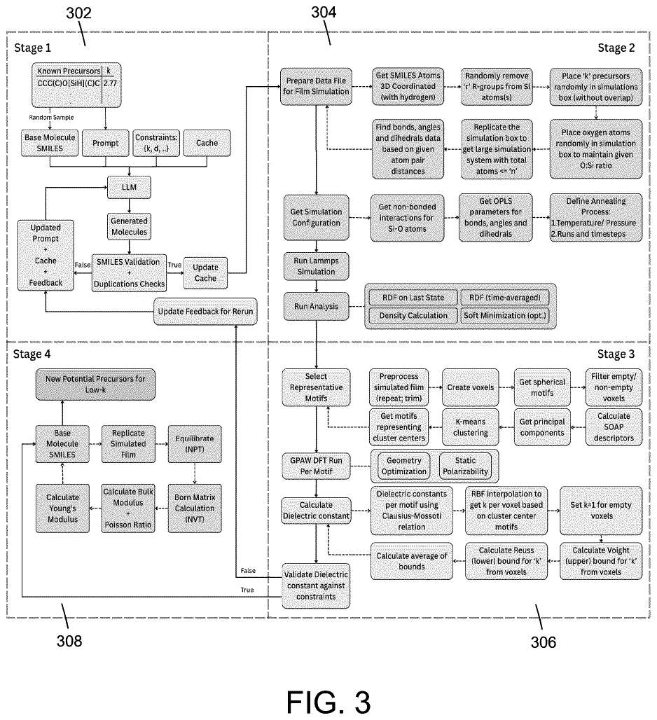

FIG. 3 illustrates an example embodiment of a four-stage generative-discriminative pipeline implemented by the AI apparatus 104. Stage 1 shows LLM-assisted precursor generation with validation and caching. Stage 2 depicts digital-twin molecular-dynamics annealing and amorphous-film formation. Stage 3 represents motif-based dielectric-constant estimation using SOAP descriptors, PCA, k-means clustering, Gaussian RBF interpolation, and Hill averaging. Stage 4 illustrates mechanical-property computation using Born-matrix analysis and DFPT corrections. The feedback module 210 aggregates results and updates the training dataset, forming an iterative learning cycle that repeats until convergence on target film properties

At the first stage 302, the generative module 202 produces an initial population of candidate precursor molecules. An LLM trained on chemical and materials corpora may receive a prompt describing target dielectric constants, desired elemental composition, or process constraints and output molecular structures in Simplified Molecular Input Line Entry System (SMILES) (a standardized text notation that encodes a molecule's structure using short ASCII strings) or graph form.

To ensure novelty and avoid redundancy, the system maintains a cache of previously generated molecules and performs duplicate filtering. Each new candidate is validated using a chemistry toolkit such as RDKit to confirm valency and stability. The valid precursors are stored with metadata defining their intended deposition route (e.g., PECVD).

In some embodiments, the LLM is fine-tuned with reinforcement learning from discriminative feedback so that future generations emphasize structural motifs correlated with low polarizability or favorable cross-linking. Conditional property vectors may be concatenated with molecular embeddings in a CDVAE to enable property-guided generation.

At the second stage 304, selected precursors are provided to digital-twin simulation module 204, which constructs and relaxes a virtual thin film representing deposition from those precursors. In one implementation, the digital-twin simulation module 204 invokes a molecular-dynamics engine such as Large-scale Atomic/Molecular Massively Parallel Simulator (LAMMPS) to perform simulated annealing across a multi-temperature schedule—for example, heating to ≈9000 K, cooling through 6000 K and 3000 K, and finally equilibrating near 300 K.

During this process, the system randomly places oxygen or dopant atoms to maintain a target stoichiometric ratio (e.g., O:Si), replicates the simulation cell along x-, y-, and z-axes, and evaluates resulting film density. The simulation captures surface reactions, precursor fragmentation, and densification dynamics analogous to PECVD.

The resulting amorphous film structure is stored in a data repository for property computation. Digital-twin simulation module 204 may further invoke motif-clustering module 206 to segment the film into local atomic environments for subsequent property analysis.

At stage three 306, the motif-clustering module 206 and the discriminative evaluation module 208 cooperate to estimate the dielectric constant of the simulated film. Each voxelized atomic environment is represented by a (SOAP) descriptor. Principal-component analysis (PCA) is first applied to reduce dimensionality, followed by k-means clustering to identify representative motifs that capture the diversity of the film's local bonding configurations.

For each representative motif, the digital-twin performs high-accuracy electronic-structure calculations to obtain local polarizability tensors. Gaussian radial-basis-function (RBF) interpolation is then used to reconstruct the continuous dielectric field across the film volume from these discrete motif values. The overall film dielectric constant k is computed as the Hill average,

$${k}_{film} = \frac{{k}_{Voigt} + {k}_{Reuss}}{2} ,$$

where k Voigt and k Reuss represent the upper and lower mechanical bounds derived from the motif data. This method provides an efficient and physically interpretable estimate of the bulk dielectric constant without requiring full DFT calculations of the entire amorphous film.

The fourth stage 308 computes mechanical properties such as bulk modulus, Poisson ratio, and Young's modulus using molecular-dynamics simulations. Digital-twin simulation module 204 applies incremental strain perturbations and evaluates the resulting stress tensor to obtain the Born matrix. The mechanical moduli are extracted from this matrix using linear-elastic relationships and may be corrected through DFPT calculations performed by the vibrational-analysis module 214. These mechanical metrics, together with the dielectric constants, characterize each film's suitability for low-k applications.

In one embodiment, the feedback and retraining module 210 aggregates results from the four stages to refine both generative and discriminative models. A multi-objective reward function is computed as a weighted combination of dielectric deviation from the target value, mechanical robustness penalty, and chemical feasibility score. Candidates meeting or surpassing desired thresholds are labeled as positive examples, while poor performers are treated as negative examples. Both are re-inserted into the training dataset maintained by the system-control module 216.

The generative model's parameters are updated through gradient descent or reinforcement learning, and the discriminative model is retrained on the expanded dataset. The external-knowledge integration module 212 may concurrently extract additional data from scientific or patent literature to augment the corpus. The updated models then initiate the next generation cycle.

The pipeline constitutes an active-learning loop in which data from simulation and model prediction continuously reinforce one another. With each iteration, the latent-space distribution of the generative model becomes more constrained around promising chemical motifs, and the discriminative model's uncertainty decreases. Convergence is detected when successive cycles produce marginal improvement in target-property reward. The final output of the system is a ranked list of precursor molecules and simulated process parameters predicted to yield thin films with optimal dielectric and mechanical properties.

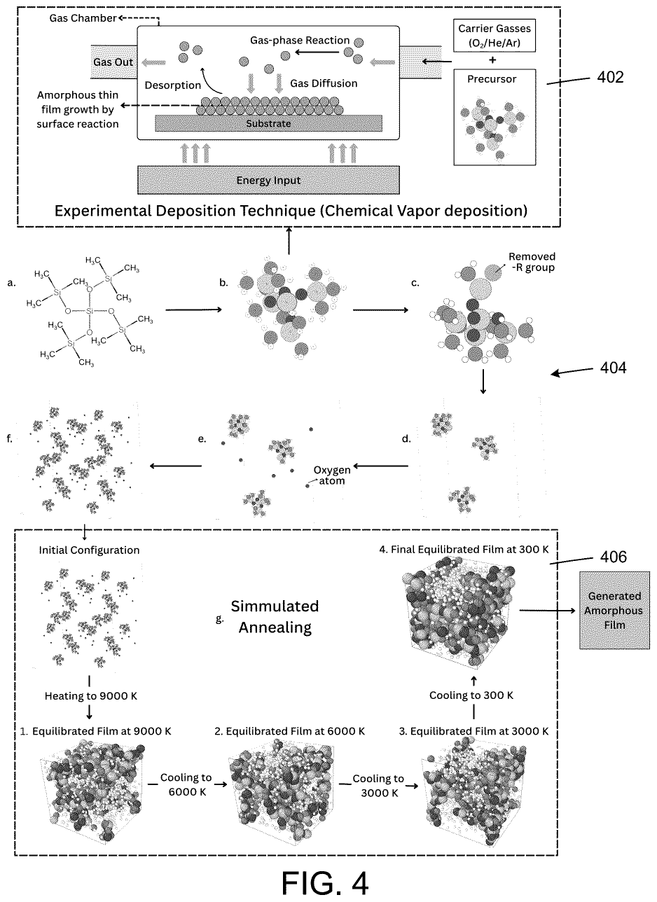

FIG. 4 illustrates an example embodiment showing correspondence between an experimental thin-film deposition technique and its digital-twin simulation as implemented by the AI apparatus 104. The upper portion of the diagram depicts PECVD system used for fabricating low-k dielectric films, while the lower portion represents the simulated amorphous-film generation process performed by the digital-twin simulation module 204.

In the experimental PECVD process 402 shown in FIG. 4, one or more precursor gases—such as organosilicates, siloxanes, or silsesquioxanes—are introduced into a vacuum reaction chamber. An RF plasma is generated between electrodes to dissociate the precursor molecules into reactive fragments. These fragments migrate toward a heated substrate and undergo surface adsorption, diffusion, and polymerization to form a solid film. The film's density, porosity, and stoichiometry depend on controllable process parameters including plasma power, pressure, gas flow rate, and substrate temperature. By adjusting these parameters and the precursor chemistry, the resulting dielectric constant (k) and mechanical strength of the film can be tuned. Higher plasma energy produces denser, higher-k layers, whereas reduced power yields more porous, lower-k structures.

The digital-twin simulation 404 reproduces these physical mechanisms in silico. Candidate precursor molecules proposed by generative module 202 are converted into atomistic configurations and placed within a simulation cell representing the deposition chamber. A molecular-dynamics engine—for example, LAMMPS—executes a multi-stage thermal-annealing schedule 406 that mimics plasma-induced fragmentation and surface reorganization. The simulated film is heated to approximately 9000 K, sequentially cooled through intermediate stages at 6000 K and 3000 K, and finally equilibrated near 300 K. During annealing, oxygen atoms are inserted to preserve an intended O:Si stoichiometric ratio, ensuring chemical realism consistent with the PECVD process.

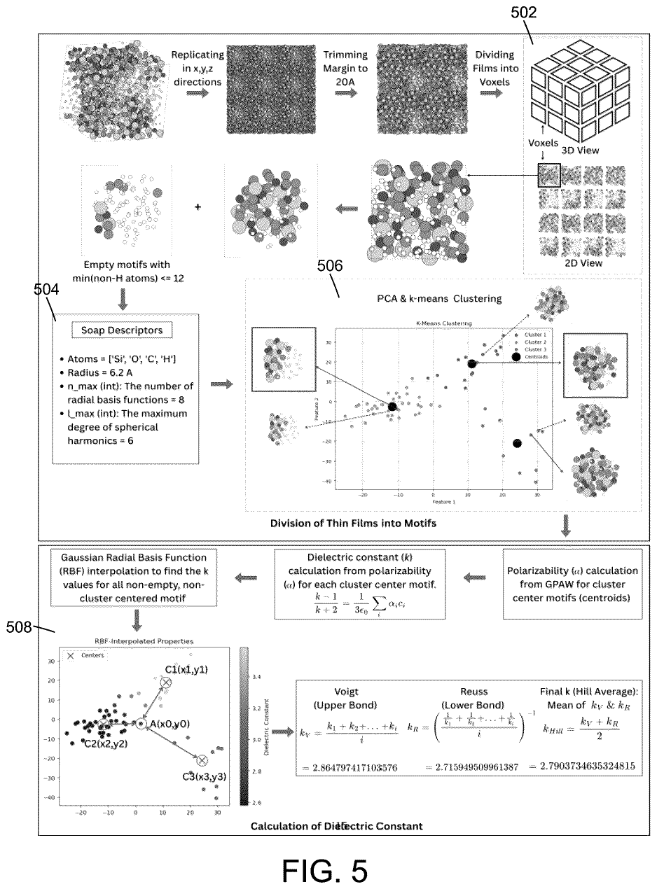

After equilibration, the simulation cell is replicated along the x-, y-, and z-axes to produce a statistically representative amorphous-film volume. The digital-twin simulation module 204 computes density and porosity to verify that the simulated microstructure corresponds to experimentally observed thin films. The resulting relaxed atomic configuration forms the digital-twin representation of the deposited layer, which is passed to the motif-clustering module 206 for dielectric-property analysis, e.g., as described in FIG. 5. In one embodiment, the digital-twin simulation employs a simulated-annealing molecular-dynamics schedule mirroring PECVD thermal cycling, including sequential cooling from approximately 9000 K to 300 K.

FIG. 4 demonstrates the direct mapping between experimental PECVD deposition and its digital-twin computational analog: plasma chemistry corresponds to high-temperature molecular-dynamics annealing, substrate temperature maps to simulation equilibration conditions, and measured film density corresponds to computed atomic-packing metrics. By aligning physical and simulated parameters, the AI apparatus 104 provides a validated, data-driven proxy for experimental deposition, enabling accurate prediction of thin-film properties and closed-loop optimization of both precursor chemistry and process conditions.

Upon completion of the simulated annealing and replication procedures illustrated in FIG. 4, the resulting amorphous thin-film structure serves as input to the next stage of the pipeline. At this point, the digital-twin simulation module 204 transfers the equilibrated atomic configuration to the motif-clustering module 206, which performs data reduction and structural analysis to identify representative atomic motifs. These motifs capture the diverse local bonding geometries present within the film—such as Si—O cages, cross-linked siloxane rings, or carbon-rich pore regions—and provide the foundation for estimating the film's electrical properties. The outputs of the motif-clustering module 206 are then evaluated by the discriminative evaluation module 208 to compute dielectric performance metrics. This sequence is depicted in FIG. 5.

FIG. 5 illustrates an example implementation of the motif-clustering and dielectric-estimation stage within the AI apparatus 104. FIG. 5 depicts how the motif-clustering module 206 and the discriminative evaluation module 208 cooperate to compute the dielectric properties of a simulated amorphous thin film generated by the digital-twin simulation module 204.

In one embodiment, shown in FIG. 5, the equilibrated film structure is divided into a plurality of three-dimensional volumetric elements 502, or voxels, each containing a localized atomic neighborhood. For every voxel, the system computes a SOAP descriptor 504 that encodes rotation- and translation-invariant information about local coordination geometry. These SOAP vectors collectively form a high-dimensional representation of the film's atomic landscape.

Next, PCA 506 is applied to reduce descriptor dimensionality and highlight dominant variations in local structure. The reduced feature vectors are grouped using k-means clustering (or an equivalent unsupervised method) to identify a finite number of representative motifs, each representing a statistically distinct bonding environment. The centroids of these clusters are designated as motif prototypes that typify recurring structural patterns across the film.

For each motif prototype, the system performs a localized electronic-structure or molecular-dynamics calculation 508 to determine the local dielectric tensor or polarizability. The resulting discrete property values are interpolated across the film volume using a Gaussian RBF model, reconstructing a continuous dielectric field from motif-level data.

The discriminative evaluation module 208 then aggregates the interpolated results and computes an effective bulk dielectric constant using the Hill-averaging approach, combining the Voigt (upper) and Reuss (lower) bounds according to:

$${k}_{film} = \frac{{k}_{Voigt} + {k}_{Reuss}}{2} .$$

This motif-based dielectric-estimation technique yields physically interpretable dielectric values while reducing computational cost by several orders of magnitude compared with full density-functional-theory simulations. As shown in FIG. 5, the outputs of this stage—local motif dielectric tensors, interpolated dielectric maps, and global averaged constants—are forwarded to the feedback and retraining module 210 for use in subsequent learning iterations of the generative-discriminative pipeline.

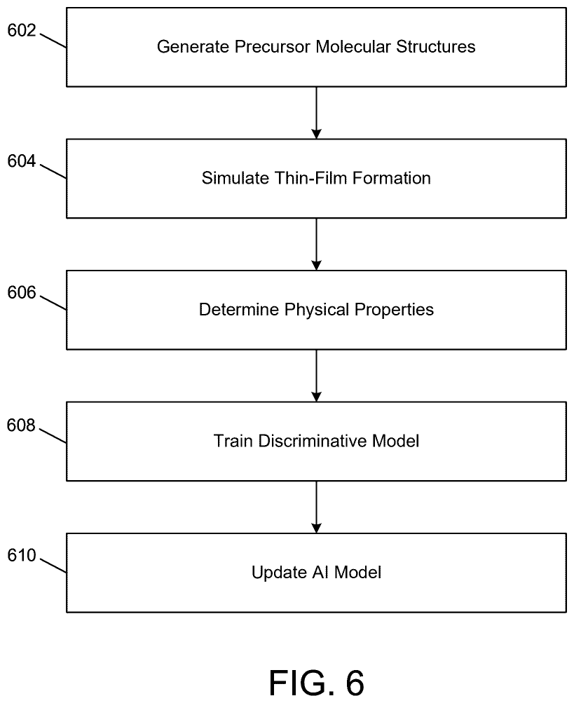

FIG. 6 depicts one embodiment of a method for generative-discriminative artificial-intelligence framework for digital-twin discovery of thin-film materials. In one embodiment, the method may be performed by an information handling device, an AI apparatus 104, a generative module 202, a digital-twin simulation module 204, a motif clustering module 206, a discriminative evaluation module 208, a feedback and retraining module 210, an external knowledge integration module 212, a vibrational analysis module 214, and/or a system control module 216.

In one embodiment, the method begins and generates 602 one or more precursor molecular structures that are candidates for forming a thin film. The generation may employ a CDVAE, a diffusion model, or a large-language model trained on chemical corpora. Each generated structure is validated for chemical stability and may be represented as a SMILES string or molecular graph. The goal of this stage is to explore chemical design space conditioned on target dielectric or mechanical properties.

In one embodiment, the method simulates 604 thin-film formation. The generated precursors are transmitted to the digital-twin simulation model, which emulates a thin-film deposition process such as PECVD. The simulator models precursor dissociation, surface adsorption, and film growth using molecular-dynamics or quantum-mechanical methods. This stage outputs an atomistic representation of the resulting amorphous or polycrystalline film.

In one embodiment, the method determines 606 physical properties of the simulated thin-film formation. Results of the simulation are analyzed to compute one or more physical properties of the thin film, including dielectric constant, density, hardness, and elastic modulus. These properties may be derived directly from molecular-dynamics trajectories or from motif-based clustering and interpolation, e.g., performed by the discriminative evaluation module 208. The determined values are compared with predefined target dielectric characteristics.

In one embodiment, the method trains 608 a discriminative learning model using the computed physical properties from the digital-twin simulation—such as dielectric constant, density, hardness, and elastic modulus. The discriminative model (e.g., a graph neural network or random-forest regressor) receives as input a feature representation of the simulated film or its representative motifs (e.g., SOAP descriptors, voxel-level embeddings, or motif-level polarizabilities). During training, the model adjusts its internal weights to minimize prediction error between its predicted and simulated (or measured) thin-film properties.

Through iterative retraining using feedback data, the discriminative model learns the quantitative relationships between local atomic structure, process parameters, and macroscopic film performance. Once trained, the discriminative model can accurately infer target properties—such as dielectric constant or mechanical modulus—directly from structural or process descriptors, without performing a full molecular-dynamics or density-functional-theory simulation. This enables rapid property prediction and active learning in the generative-discriminative loop, allowing the AI apparatus 104 to evaluate and optimize new film candidates orders of magnitude faster than full physics-based computation.

In one embodiment, the method updates 610 generative AI models. For instance, the method calculates a reward or loss value based on deviations between the simulated properties and target specifications. The parameters of the generative model are then updated accordingly. This iterative update refines the model's latent-space distribution so that subsequent generations yield precursors predicted to produce improved thin-film properties. The method repeats until convergence on candidate materials meeting desired dielectric and mechanical criteria.

The flowchart thus represents a closed-loop generative-discriminative framework in which artificial-intelligence-driven molecular design is continuously informed by physics-based digital-twin simulations. Through successive iterations, the AI apparatus 104 autonomously discovers and optimizes precursor materials predicted to form thin films having target dielectric characteristics

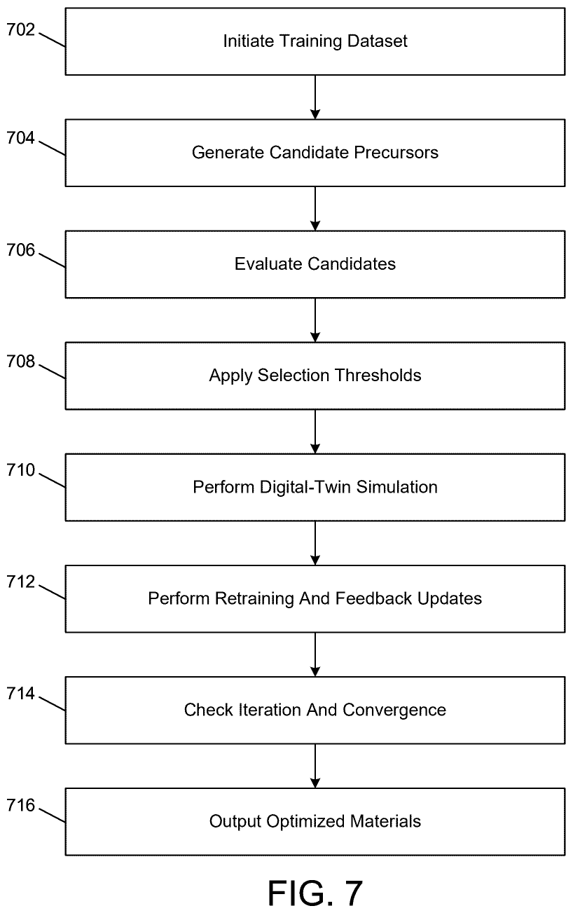

FIG. 7 depicts one embodiment of a method for generative-discriminative artificial-intelligence framework for digital-twin discovery of thin-film materials. In one embodiment, the method may be performed by an information handling device, an AI apparatus 104, a generative module 202, a digital-twin simulation module 204, a motif clustering module 206, a discriminative evaluation module 208, a feedback and retraining module 210, an external knowledge integration module 212, a vibrational analysis module 214, and/or a system control module 216.

In one embodiment, the method begins and initializes 702 a training dataset. The initial training dataset may contain both favorable (“good”) and unfavorable (“bad”) material examples. Each entry includes precursor structure data, associated deposition conditions, and measured or simulated dielectric and mechanical properties. This dataset establishes the baseline used to train generative and discriminative models.

In one embodiment, the method generates 704 new molecular candidates conditioned on target dielectric characteristics. Generation may use a conditional variational autoencoder, diffusion model, or LLM that outputs chemical formulas or SMILES strings consistent with synthesis constraints. Heuristic atom-substitution or molecular-fragment replacement may also be employed to create chemically diverse variants. All generated candidates are validated for structural stability and stored for evaluation.

In one embodiment, the method evaluates 706 candidates with a discriminative model. Each candidate is processed to predict dielectric constants, density, hardness, and other relevant properties. Fast classifiers such as random-forest regressors may serve as preliminary filters, while higher-fidelity graph-neural-network models refine the predictions.

In one embodiment, the method applies 708 selection thresholds. Predicted results are compared with predefined target criteria. Candidates whose scores exceed the acceptance threshold are labeled as potential positives and forwarded for detailed digital-twin simulation; those below the threshold are rejected or stored as negative examples to improve model discrimination. This filtering reduces computational load while maintaining a balanced dataset of successes and failures.

In one embodiment, the method performs 710 digital-twin simulation. Accepted candidates are transferred to a digital-twin simulation model, which emulates thin-film formation under representative deposition conditions (e.g., PECVD). Simulated films are produced and structural and physical properties are computed such as dielectric constant, density, and mechanical modulus. Results are added to the property database for feedback analysis.

In one embodiment, the method retrains 712 using feedback. The method computes a multi-objective reward function reflecting deviations between simulated and target properties. The generative and discriminative model parameters are updated using these rewards, and the new information—both accepted and rejected candidates—is incorporated into the training dataset.

In one embodiment, the method performs 714 an iteration and convergence check. The method determines whether performance metrics, such as prediction accuracy or average deviation from target dielectric constant, satisfy a convergence threshold. If convergence has not been reached, the method returns to generate new candidates using the updated models. The loop continues until convergence or until a sufficient number of materials meet design targets.

In one embodiment, the method outputs 716 optimized materials. When convergence is achieved, the process outputs a ranked list of precursor molecules and deposition parameters predicted to yield thin films with optimal dielectric and mechanical characteristics. These results can be transferred directly to experimental synthesis or stored for future retraining.

FIG. 7 represents a complete active-learning pipeline linking data initialization, generative design, discriminative evaluation, digital-twin simulation, and feedback retraining in a continuous loop. Through successive iterations, the AI apparatus 104 autonomously refines its predictive accuracy and converges on precursor and process combinations that achieve target dielectric performance.

In one embodiment, an apparatus is configured to generate, using a generative artificial intelligence model, one or more precursor molecular structures for forming a thin film; simulate, using a digital-twin model of a thin-film deposition process, formation of the thin film from the one or more precursor molecular structures; determine, from results of the simulation, one or more physical properties of the thin film; train a discriminative model using the one or more physical properties of the thin film, wherein the discriminative model learns to predict thin-film properties without requiring full simulation; and update the generative artificial intelligence model based on the one or more physical properties of the thin film or predictions generated by the discriminative model to iteratively improve discovery of materials having target dielectric characteristics.

In one embodiment, the digital-twin model comprises a physics-based proxy of a PECVD process. In one embodiment, the digital-twin model of the thin-film deposition process is implemented as a simulated annealing method in which random substitution of atoms within a simulation cell is used as a proxy for precursor gas flow in a PECVD process. In one embodiment, the simulated annealing method comprises heating and cooling the thin film across a temperature range to model film densification and amorphous-structure formation.

In one embodiment, the simulation comprises dividing an atomic model of the thin film into three-dimensional voxels, clustering the voxels into motifs, and performing physical simulations on representative motifs to estimate the physical properties. In one embodiment, determining the one or more physical properties comprises computing dielectric constant, hardness, density, and elastic modulus of the thin film.

In one embodiment, the generative artificial intelligence model comprises an LLM trained on chemical and materials corpora. In one embodiment, the LLM is fine-tuned using reinforcement learning from simulation feedback to bias precursor generation toward structures having target dielectric constants.

In one embodiment, the apparatus is configured to use the discriminative model as a proxy predictor to estimate thin-film properties for additional precursor candidates without executing full simulations. In one embodiment, the discriminative model comprises a random-forest regressor or a graph-neural-network model trained to approximate outputs of the digital-twin model.

In one embodiment, the apparatus is configured to retrain the generative model using a reward based on a deviation between predicted and simulated dielectric constants. In one embodiment, the simulation further includes modeling vibrational effects of the thin film using DFPT.

In one embodiment, the apparatus is configured to incorporate external domain knowledge using a large language model trained on patent and scientific literature describing dielectric materials. In one embodiment, the digital-twin model computes dielectric and mechanical properties of the thin film using motif-based DFT analysis performed on representative atomic motifs identified from the thin film.

In one embodiment, a method is configured for generating, using a generative artificial intelligence model, one or more precursor molecular structures for forming a thin film; simulating, using a digital-twin model of a thin-film deposition process, formation of the thin film from the one or more precursor molecular structures; determining, from results of the simulation, one or more physical properties of the thin film; training a discriminative model using the one or more physical properties of the thin film, wherein the discriminative model learns to predict thin-film properties without requiring full simulation; and updating the generative artificial intelligence model based on the one or more physical properties of the thin film or predictions generated by the discriminative model to iteratively improve discovery of materials having target dielectric characteristics.

In one embodiment, a computer program product is embodied on a non-transitory computer-readable medium comprising instructions that, when executed by a processor, cause the processor to perform operations comprising generating, using a generative artificial intelligence model, one or more precursor molecular structures for forming a thin film; simulating, using a digital-twin model of a thin-film deposition process, formation of the thin film from the one or more precursor molecular structures; determining, from results of the simulation, one or more physical properties of the thin film; training a discriminative model using the one or more physical properties of the thin film, wherein the discriminative model learns to predict thin-film properties without requiring full simulation; and updating the generative artificial intelligence model based on the one or more physical properties of the thin film or predictions generated by the discriminative model to iteratively improve discovery of materials having target dielectric characteristics.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom very large scale integrated (“VLSI”) circuits or gate arrays, off-the-shelf semiconductors such as logic chips, transistors, or other discrete components. A module may also be implemented in programmable hardware devices such as a field programmable gate array (“FPGA”), programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some embodiments, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Many of the functional units described in this specification have been labeled as modules, to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods, and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C. As used herein, “a member selected from the group consisting of A, B, and C and combinations thereof” includes only A, only B, only C, a combination of A and B, a combination of B and C, a combination of A and C or a combination of A, B and C.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus, comprising:
at least one memory; and
at least one processor coupled with the at least one memory and configured to cause the apparatus to:
generate, using a generative artificial intelligence model, one or more precursor molecular structures for forming a thin film;
simulate, using a digital-twin model of a thin-film deposition process, formation of the thin film from the one or more precursor molecular structures;
determine, from results of the simulation, one or more physical properties of the thin film;
train a discriminative model using the one or more physical properties of the thin film, wherein the discriminative model learns to predict thin-film properties without requiring full simulation; and
update the generative artificial intelligence model based on the one or more physical properties of the thin film or predictions generated by the discriminative model to iteratively improve discovery of materials having target dielectric characteristics.

2. The apparatus of claim 1, wherein the digital-twin model comprises a physics-based proxy of a plasma-enhanced chemical vapor deposition (PECVD) process.

3. The apparatus of claim 1, wherein the digital-twin model of the thin-film deposition process is implemented as a simulated annealing method in which random substitution of atoms within a simulation cell is used as a proxy for precursor gas flow in a plasma-enhanced chemical vapor deposition (PECVD) process.

4. The apparatus of claim 3, wherein the simulated annealing method comprises heating and cooling the thin film across a temperature range to model film densification and amorphous-structure formation.

5. The apparatus of claim 1, wherein the simulation comprises dividing an atomic model of the thin film into three-dimensional voxels, clustering the voxels into motifs, and performing physical simulations on representative motifs to estimate the physical properties.

6. The apparatus of claim 1, wherein determining the one or more physical properties comprises computing dielectric constant, hardness, density, and elastic modulus of the thin film.

7. The apparatus of claim 1, wherein the generative artificial intelligence model comprises a large language model (LLM) trained on chemical and materials corpora.

8. The apparatus of claim 7, wherein the LLM is fine-tuned using reinforcement learning from simulation feedback to bias precursor generation toward structures having target dielectric constants.

9. The apparatus of claim 1, wherein the at least one processor is configured to cause the apparatus to use the discriminative model as a proxy predictor to estimate thin-film properties for additional precursor candidates without executing full simulations.

10. The apparatus of claim 9, wherein the discriminative model comprises a random-forest regressor or a graph-neural-network model trained to approximate outputs of the digital-twin model.

11. The apparatus of claim 1, wherein the at least one processor is configured to cause the apparatus to retrain the generative model using a reward based on a deviation between predicted and simulated dielectric constants.

12. The apparatus of claim 1, wherein the simulation further includes modeling vibrational effects of the thin film using density functional perturbation theory (DFPT).

13. The apparatus of claim 1, wherein the at least one processor is configured to cause the apparatus to incorporate external domain knowledge using a large language model trained on patent and scientific literature describing dielectric materials.

14. The apparatus of claim 10, wherein the digital-twin model computes dielectric and mechanical properties of the thin film using motif-based density-functional-theory (DFT) analysis performed on representative atomic motifs identified from the thin film.

15. A method, comprising:
generating, using a generative artificial intelligence model, one or more precursor molecular structures for forming a thin film;
simulating, using a digital-twin model of a thin-film deposition process, formation of the thin film from the one or more precursor molecular structures;
determining, from results of the simulation, one or more physical properties of the thin film;
training a discriminative model using the one or more physical properties of the thin film, wherein the discriminative model learns to predict thin-film properties without requiring full simulation; and
updating the generative artificial intelligence model based on the one or more physical properties of the thin film or predictions generated by the discriminative model to iteratively improve discovery of materials having target dielectric characteristics.

16. The method of claim 15, wherein the digital-twin model comprises a physics-based proxy of a plasma-enhanced chemical vapor deposition (PECVD) process.

17. The method of claim 15, wherein the digital-twin model of the thin-film deposition process is implemented as a simulated annealing method in which random substitution of atoms within a simulation cell is used as a proxy for precursor gas flow in a plasma-enhanced chemical vapor deposition (PECVD) process.

18. The method of claim 17, wherein the simulated annealing method comprises heating and cooling the thin film across a temperature range to model film densification and amorphous-structure formation.

19. The method of claim 15, wherein the simulation comprises dividing an atomic model of the thin film into three-dimensional voxels, clustering the voxels into motifs, and performing physical simulations on representative motifs to estimate the physical properties.

20. A computer program product embodied on a non-transitory computer-readable medium comprising instructions that, when executed by a processor, cause the processor to perform operations comprising:
generating, using a generative artificial intelligence model, one or more precursor molecular structures for forming a thin film;
simulating, using a digital-twin model of a thin-film deposition process, formation of the thin film from the one or more precursor molecular structures;
determining, from results of the simulation, one or more physical properties of the thin film;
training a discriminative model using the one or more physical properties of the thin film, wherein the discriminative model learns to predict thin-film properties without requiring full simulation; and
updating the generative artificial intelligence model based on the one or more physical properties of the thin film or predictions generated by the discriminative model to iteratively improve discovery of materials having target dielectric characteristics.
